(19) United States
(12) Patent Application Publication
(10) Pub. No.: US 2014/0086853 A1
(43) Pub. Date: Mar. 27, 2014

(54) Personal Compositions With Silicone Emulsifier-Free Natural Emulsifier System

(71) Applicant: Catherine Le Pham, Minneapolis, MN (US)

(72) Inventor: Catherine Le Pham, Minneapolis, MN (US)

(21) Appl. No.: 13/627,810

(22) Filed: Sep. 26, 2012

(51) Int. Cl. A61K 8/60; A61Q 7/00; A61Q 19/02; A61Q 19/06; A61K 8/06; A61Q 17/04

(52) U.S. Cl. CPC A61K 8/602; A61K 8/60; A61K 8/062; A61Q 17/04; A61Q 19/02; A61Q 19/06; A61Q 7/00
USPC 424/59; 514/777; 424/729; 514/263.34; 424/728; 424/758; 424/765; 424/727; 424/725; 514/1.1; 424/62

(57) ABSTRACT

The present invention relates to a personal care composition comprising at least one sucrose fatty acid ester and at least one nonionic surfactant, wherein the composition is substantially free of silicone emulsifiers. The personal care compositions are particularly suitable for conditioning and moisturization applications.

### CROSS-REFERENCE TO RELATED APPLICATIONS

This application claims priority from U.S. Provisional Application No. 61/541,747 filed Sep. 30, 2011.

### FIELD OF THE INVENTION

The present invention relates to personal care compositions comprising an oil-in-water emulsifier system. The emulsifier system may achieve a low-soaping index in personal care compositions which are free of silicone emulsifiers.

### BACKGROUND OF THE INVENTION

Volatile silicones and dimethicones are widely used as carriers or diluents for personal care and cosmetic applications. They enhance spreading and lubricating properties, evaporate quickly from the skin without cooling or stinging it, and leave it feeling pleasantly dry and smooth. They lubricate spray valves and roll-on applicators, ensure stick compositions spread smoothly, and prevent soaping and whitening during application. Therefore, silicones have traditionally been used in personal care compositions to provide one or more of the aforementioned benefits.

Consumers desire personal care compositions which are easy to apply to skin and dislike compositions which form dry or filmy residues. In order to design products with high spreadability, a low formulation viscosity is preferable. In addition, consumers are becoming more aware of the safety of the ingredients used in their cosmetic products. In particular, the use of silicone and silicone-based products has drawn negative attention from the media. Silicone and its derivatives have been used as the primary carrier, wetting agent or coating agent in a number of commercially available cosmetic and personal care dispersions that formulators have come to depend on. However, silicone and its derivatives, though widely used in cosmetics for many years, are once again undergoing scrutiny for their safety as well as environmental impact. In view of this situation, silicone and its derivatives may fall out of favor with consumers, requiring formulators to find suitable substitutes if they are to continue providing dispersion-based cosmetic and personal care products. Accordingly, there is a need for formulations which remain stable and low-soaping even in formulations which are free of silicone emulsifiers.

### SUMMARY OF THE INVENTION

The present invention relates to a personal care composition comprising an oil-in-water emulsifier system comprising at least one sucrose fatty acid ester and at least one nonionic surfactant, said system being substantially free of silicone emulsifiers.

### DETAILED DESCRIPTION

While the specification concludes with claims that particularly point out and distinctly claim the invention, it is believed the present invention will be better understood from the following description.

All percentages, parts and ratios are based upon the total weight of the compositions of the present invention, unless otherwise specified. All such weights as they pertain to listed ingredients are based on the active level, and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The term “oil-soluble” as used herein, means that the material is soluble in oil. In general, the material should be soluble at 25° C. at a concentration of at least 0.1% by weight of the oil solvent, preferably at least 1%, more preferably at least 5%, most preferably at least 15%.

The term “oil-insoluble” as used herein, means that a material is not soluble in oil. Thus, the material is not miscible with oil.

The term “polymer” as used herein shall include materials whether made by polymerization of one type of monomer or made by two (i.e., copolymers) or more types of monomers.

The term “personal care composition” as used herein, refers to compositions intended for topical application to the skin or hair and specifically excludes those compositions that are directed primarily to other applications such as hard surface cleansing, fabric or laundry cleansing, and similar other applications not intended primarily for topical application to the hair or skin. Examples of personal care compositions of the present invention may include but are not limited to lotions, sunless tanner, sunscreen lotions, antiperspirants, deodorants, shave creams, face creams, color cosmetics, lipstick, mascara, blush, foundation, nail polish, and the like. Rinse off compositions such as shampoo, conditioning shampoo, body wash, moisturizing body wash, shower gels, skin cleansers, cleansing milks, hair and body wash, in shower body moisturizer, pet shampoo, shaving preparations and cleansing compositions used in conjunction with a disposable cleansing cloth are also suitable.

The term “silicone emulsifier” as used herein means a silicone based ingredient which serves the purpose of suspending or dispersing other components of a personal care composition. Silicone emulsifiers, as defined herein, specifically do not include silicone-based ingredients which act as emollients, opacifiers, particulates, and the like, which are not intended to emulsify the composition.

The term “solid particle” as used herein means a particle that is not a liquid or a gas.

The phrase “substantially free of” as used herein, unless otherwise specified means that the composition comprises less than about 5%, preferably less than about 3%, more preferably less than about 1% and most preferably less than about 0.1% of the stated ingredient. The term “free of” as used herein means that the composition comprise 0% of the stated ingredient that is the ingredient has not been added to the composition, however, these ingredients may incidentally form as a byproduct or a reaction product of the other components of the composition.

The term “water-soluble” as used herein, means that the polymer is soluble in water in the present composition. In general, the polymer should be soluble at 25° C. at a concentration of at least 0.1% by weight of the water solvent, preferably at least 1%, more preferably at least 5%, most preferably at least 15%.

The term “water-insoluble” as used herein, means that a compound is not soluble in water in the present composition. Thus, the compound is not miscible with water.

### Personal Care Composition

The personal care composition of the present invention is an oil-in-water emulsion comprising a continuous water phase and a discontinuous oil phase. Suitable emulsions may have a wide range of viscosities, depending on the desired product form. In certain embodiments, the personal care composition may have a viscosity of from about 1,000 cps (centipoise) to about 1,000,000 cps, or from about 5,000 cps to about 500,000 cps, or from about 10,000 cps to about 200,000 cps, or from about 15,000 cps to about 75,000 cps. For example, viscosities may be measured on a Brookfield viscometer using a T-C bar spindle with a heliopath setting at 5 rpm at 25° C. In particular embodiments, the personal care composition has a pH of from about 3 to about 9, or from about 4 to about 7.

The personal care composition may comprise at least about 2% of an oil phase. The personal care composition may comprise from about 2% to about 75%, or from about 5% to about 35%, or from about 10% to about 30%, by weight of the composition, of an oil phase. The oil phase component is preferably essentially silicone-free, ie contains no more than about 10%, preferably no more than about 5% by weight of silicon-based materials. It will be understood that the oil phase may contain small levels (eg. up to about 25%, preferably 10%) of oil phase soluble emulsifier ingredients.

Suitable oil phase components for use herein include, for example, optionally hydroxy-substituted C.sub.8-C.sub.50 unsaturated fatty acids and esters thereof, C.sub.1-C.sub.24 esters of C.sub.8-C.sub.30 saturated fatty acids such asisopropyl myristate, cetyl palmitate and octyldodecylmyristate (Wickenol 142), beeswax, saturated and unsaturated fatty alcohols such as behenyl alcohol and cetyl alcohol, hydrocarbons such as mineral oils, petrolatum and squalane, fatty sorbitan esters, lanolin and lanolin derivatives, animal and vegetable triglycerides such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grapeseed oil, and sunflower seed oil and C₁-C₂₄ esters of dimer and trimer acids such as diisopropyl dimerate, diisostearylmalate, diisostearyidimerate and triisostearyltrimerate. Of the above, highly preferred are the mineral oils, petrolatums, unsaturated fatty acids and esters thereof and mixtures thereof.

The personal care composition may comprise from about 25% to about 98%, or from about 65% to about 95%, or from about 70% to about 90%, by weight of the composition, of a water phase. In some embodiments, the water phase may be comprised entirely of water. In other embodiments, the water phase may comprise components other than water (i.e., non-water components), including, but not limited to, water-soluble moisturizing agents, conditioning agents, anti-microbials, humectants, and/or other water-soluble skin care actives, to impart an increased benefit to the keratinous tissue. In one embodiment, the water phase comprises a humectant such as glycerin and/or other polyols. In a particular embodiment, the personal care composition may be substantially water-free.

In a particular embodiment, the personal care composition includes an oil-in-water emulsifier system comprising at least one sucrose fatty acid ester and at least one nonionic surfactant. The sucrose fatty acid ester is preferably selected from the group consisting of sucrose dilaurate, sucrose distearate, sucrose hexaerucate; sucrose hexaoleate/hexapalmitate/hexastearate, sucrose hexapalmitate, sucrose laurate; sucrose myristate, sucrose oleate, sucrose palmitate, sucrose pentaerucate, sucrose polybehenate, sucrose polylaurate, sucrose polylinoleate, sucrose polyoleate, sucrose polystearate, sucrose ricinoleate, sucrose stearate, sucrose tetraisostearate, sucrose tribehenate, sucrose trilaurate, sucrose tristearate, sucrose cocoate, sucrose polycottonseedate, sucrose polypalmate, sucrose polysoyate, and mixtures thereof. In a more preferred embodiment, the sucrose fatty acid ester is sucrose stearate. In a particular embodiment, the nonionic surfactant is selected from the group consisting of methyl glucose sesquisterate, PPG-20 methyl glucose ether, PPG-20 methyl glucose ether distearate, PEG-120 methyl glucose dioleate and ethoxylated methyl glucose having from about 10 to about 20 repeating ethoxy units. In a preferred embodiment, the nonionic surfactant is methyl glucose sesquisterate.

The emulsifier system herein surprisingly avoids the soaping effect evidenced by other personal care formulations. In order to suppress soaping in many emulsifications, formulators have traditionally used silicone, or specifically, dimethicone. The emulsifier system herein is further surprising in that soaping may be reduced or eliminated in formulations which are substantially free of silicone emulsifiers. The low-soaping effect is assisted by achieving a ratio of sucrose fatty acid ester to nonionic surfactant of from about 1:3 to about 3:1. In a preferred embodiment, the ratio of sucrose fatty acid ester to nonionic surfactant is about 1:1. As used herein, the term “emulsifier system” refers to the combination of both the sucrose fatty acid ester and nonionic surfactant.

Preferably the emulsifier system has an HLB from about 1.5 to 13.0, preferably from about 3.4 to 13.0, more preferably 3.4 to about 9.5, more preferably 3.4 to about 8.0. When formulated in a personal care or cosmetic composition, the emulsifier system is present at a level ranging from about 0.1% to about 10%, more preferably from about 0.25% to about 8%, even more preferably from about 0.5% to about 7%, still even more preferably from about 1.0% to about 5%, and still even still more preferably from about 1.5% to about 4%, by weight of the personal or cosmetic care compositions.

The balance between the hydrophilic and lipophilic moieties in a surfactant molecule is used as a method of classification (hydrophile-lipophile balance, HLB). The HLB values for commonly-used surfactants are readily available in the literature (e.g., HLB Index in McCutcheon's Emulsifiers and Detergents, MC Publishing Co., 2004). For example, cocamide monoethanolamine (CMEA) is known in the art to have an HLB value of 16.8. Another way of obtaining HLB values is to estimate by calculations. The HLB system was originally devised by Griffin (J. Soc. Cosmetic Chem., 1, 311, 1949). Griffin defined the HLB value of a surfactant as the mol % of the hydrophilic groups divided by 5, where a completely hydrophilic molecule (with no non-polar groups) had an HLB value of 20. Other examples of how to calculate HLB values are described by Davies in Interfacial Phenomena, 2nd Edition, Academic Press, London, 1963 and by Lin in J. Phys. Chem. 76, 2019-2013, 1972.

### Additional Actives

The composition of the present invention may comprise one or more active ingredients. Particularly suitable actives include sugar amines, vitamin B3 compounds, vitamins, peptides, and sunscreens.

### Sugar Amine

The compositions of the present invention may comprise a sugar amine, also known as amino sugars, and their salts, isomers, tautomers and derivatives. As used herein, “sugar amine” refers to a pure sugar amine compound or a mixture of sugar amine compounds (e.g., extracts from natural sources or mixtures of synthetic materials) of synthetic or natural origin, including its isomers, tautomers, salts, and derivatives. Examples of sugar amines useful herein include glucosamine, N-acetyl glucosamine, mannosamine, N-acetyl mannosamine, galactosamine, N-acetyl galactosamine, their isomers (e.g., stereoisomers), and their salts (e.g., HCl salt). In one embodiment, the sugar amine is glucosamine, or D-glucosamine and N-acetyl glucosamine, or N-acetyl-D-glucosamine. Additionally, combinations of two or more sugar amines may be used. The composition may comprise from about 0.01% to about 15%, alternatively from about 0.1% to about 10%, and alternatively from about 0.5% to about 5%, of a sugar amine.

Vitamin B3 Compound

The compositions of the present invention may include a vitamin B3 compound. As used herein, “vitamin B3 compound” means a compound having the formula:

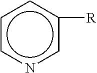

wherein R is —CONH2 (i.e., niacinamide), —COOH (i.e., nicotinic acid) or —CH₂OH (i.e., nicotinyl alcohol); derivatives thereof; and salts of any of the foregoing. Vitamin B3 compounds are particularly useful for regulating skin condition as described in U.S. Pat. No. 5,939,082. In one embodiment, the vitamin B3 compound is niacinamide. The composition may comprise from about 0.001% to about 20%, or from about 0.1% to about 10%, or from about 0.5% to about 7%, by weight of the composition, of a vitamin B3 compound.

Vitamins

The composition of the present invention may comprise one or more vitamins, for example, to provide antioxidant and/or other nutritional benefits to the skin. Herein, “vitamin” means vitamins, pro-vitamins, and their salts, isomers and derivatives. The vitamins may include water soluble vitamins, for example, nicotinic acid, C1-C18 nicotinic acid esters, and nicotinyl alcohol; B6 compounds, such as pyroxidine; and B5 compounds, such as panthenol, or “pro-B5”); and Vitamin C compounds, including ascorbyl esters of fatty acids, and ascorbic acid derivatives, for example, ascorbyl glucoside, magnesium ascorbyl phosphate, sodium ascorbyl phosphate, and ascorbyl sorbate; and mixtures thereof. The vitamins also may include those exhibiting limited solubility in water, such as Vitamin A compounds, and all natural and/or synthetic analogs of Vitamin A, including retinoids, carotenoids, and other compounds which possess the biological activity of Vitamin A; Vitamin D compounds; Vitamin E compounds, or tocopherol, including tocopherol sorbate, tocopherol acetate, other esters of tocopherol; Vitamin K compounds; and mixtures thereof. The composition may comprise from about 0.0001% to about 20%, or from about 0.01% to about 15%, or from about 0.1% to about 10%, by weight of the composition, of a vitamin.

Peptides

The compositions of the present invention may include a peptide. As used herein, “peptide” refers to peptides containing ten or fewer amino acids and their derivatives, isomers, and complexes with other species such as metal ions (e.g., copper, zinc, manganese, magnesium, and the like). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, and Cu-histidine-glycine-glycine (Cu-HGG, also known as IAMIN®). A further example includes carnosine (beta-alanine-histidine). Preferred peptides include PROMATRIXYL, comprising palmitoyl pentapeptide-3 and PALESTRINA, comprising palmitoyl dipeptide-7, both available from Croda Inc. The composition may comprise from about 1×10-6% to about 20%, or from about 1×10-5% to about 10%, or from about 1×10-4% to about 5%, by weight of the composition, of a peptide.

Sunscreens

The compositions of the subject invention may comprise one or more sunscreen actives. As used herein, “sunscreen active” refers to oil-soluble sunscreens, insoluble sunscreens, and water-soluble sunscreens. Non-limiting examples of suitable oil-soluble sunscreens are disclosed in The Cosmetic, Toiletry, and Fragrance Association's The International Cosmetic Ingredient Dictionary and Handbook, 10th Ed., Gottschalck, T. E. and McEwen, Jr., Eds. (2004), p. 2267 and pp. 2292-93 and include benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, and derivatives and mixtures thereof. Non-limiting examples of suitable insoluble sunscreens include methylene bis-benzotriazolyl tetramethylbutyl-phenol, titanium dioxide, zinc cerium oxide, zinc oxide, and derivatives and mixtures thereof. Non-limiting examples of suitable water-soluble sunscreens include phenylbenzimidazole sulfonic acid (PBSA), terephthalylidene dicamphor sulfonic acid, (Mexoryl™ SX), benzophenone-4, benzophenone-5, benzylidene camphor sulfonic acid, cinnamidopropyl-trimonium chloride, methoxycinnamido-propyl ethyldimonium chloride ether, disodium bisethylphenyl triaminotriazine stilbenedisulfonate, disodium distyrylbiphenyl disulfonate, disodium phenyl dibenzimidazole tetrasulfonate, methoxycinnamido-propyl hydroxysultaine, methoxycinnamido-propyl laurdimonium tosylate, PEG-25 PABA (p-aminobenzoic acid), polyquaternium-59, TEA-salicylate, and salts, derivatives and mixtures thereof. The composition may comprise from about 0.0001% to about 30%, or from about 0.01% to about 20%, or from about 0.1% to about 10%, by weight of the composition, of a vitamin.

Retinoid

The compositions of this invention may comprise a safe and effective amount of a retinoid. As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably selected from retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid), or mixtures thereof. In one embodiment, the retinoid is retinyl propionate. The composition may comprise from about 0.001% to about 10%, or from 0.01% to about 1%, or from about 0.01% to about 0.5%, by weight of the composition, of a retinoid.

### Other Actives

Other suitable actives include, but are not limited to, oil control agents, N-acyl amino acid compounds, tanning actives, anti-acne actives, desquamation actives, anti-cellulite actives, chelating agents, skin lightening agents, flavonoids, protease inhibitors, tyrosinase inhibitors, non-vitamin antioxidants and radical scavengers, preservatives, hair growth regulators, anti-wrinkle actives, anti-atrophy actives, minerals, phytosterols and/or plant hormones, anti-inflammatory agents, antimicrobials, and antifungals. Further suitable actives include caffeine; tea extracts, e.g. white tea extract and green tea extract; ginseng; cucumber extract; rosehip oil; date palm kernel extract; witch hazel extract; dill extract; tetrahydrocurcmin; turmerone; and other natural or botanical compounds. Many of these actives are provided in further detail in U.S. Application Publication Nos. US2006/0275237A1, US2004/0175347A1, and US2006/0263309A1.

### Optional Ingredients

Particulate Material

The compositions of the present invention may comprise from about 0.001% to about 40%, by weight of the composition, of one or more particulate materials. Non-limiting examples of suitable powders include inorganic powders (for example, iron oxides, titanium dioxides, zinc oxides, silica), organic powders, composite powders, optical brightener particles, and mixtures of any of the foregoing. These particulates can, for instance, be platelet shaped, spherical, elongated or needle-shaped, or irregularly shaped; surface coated or uncoated; porous or non-porous; charged or uncharged; and can be added to the current compositions as a powder or as a pre-dispersion. In one embodiment, the particulate material is hydrophobically coated.

Suitable organic powders particulate materials include, but are not limited, to polymeric particles chosen from the methylsilsesquioxane resin microspheres, e.g., Tospearl™ 145A, (Toshiba Silicone); microspheres of polymethylmethacrylates, e.g., Micropearl™ M 100 (Seppic); the spherical particles of crosslinked polydimethylsiloxanes, e.g., Trefil™ E 506C or Trefil™ E 505C (Dow Corning Toray Silicone); spherical particles of polyamide, e.g., nylon-12, and Orgasol™ 2002D Nat C05 (Atochem); polystyrene microspheres, e.g., Dyno Particles, sold under the name Dynospheres™, and ethylene acrylate copolymer, sold under the name FloBead™ EA209 (Kobo); aluminum starch octenylsuccinate, e.g., Dry Flo™ (National Starch); polyethylene particulates, e.g., Microthene™ FN510-00 (Equistar) and Micropoly® 220L (Micro Powders, Inc.); microspheres of polypropylene, e.g., Mattewax™ 511 (Micro Powders, Inc.); silicone resin; polymethylsilsesquioxane silicone polymer; platelet shaped powder made from L-lauroyl lysine; and mixtures thereof.

The composition of the present invention further may comprise interference pigments, including hydrophobically-modified interference pigments. Herein, “interference pigments” means thin, plate-like layered particles having two or more layers of controlled thickness. The layers have different refractive indices that yield a characteristic reflected color from the interference of typically two, but occasionally more, light reflections, from different layers of the plate-like particle. One example of interference pigments are micas layered with about 50-300 nm films of TiO2, Fe2O3, silica, tin oxide, and/or Cr2O3 and include pearlescent pigments. Interference pigments are available commercially from a wide variety of suppliers, for example, Rona (Timiron™ and Dichrona™), Presperse (Flonac™), Englehard (Duochrome™), Kobo (SK-45-R and SK-45-G), BASF (Sicopearls™) and Eckart (Prestige™). In one embodiment, the average diameter of the longest side of the individual particles of interference pigments is less than about 75 microns, and alternatively less than about 50 microns.

Non-limiting examples of suitable colorants include iron oxides, ferric ammonium ferrocyanide, manganese violet, ultramarine blue, and chromium oxide, phthalocyanine blue and green pigment, encapsulated dyes, inorganic white pigments, for example TiO2, ZnO, or ZrO2, FD&;C dyes, D&;C dyes, and mixtures thereof.

Inorganic Sunscreens

The composition further may comprise from about 0.001% to about 10%, and alternatively from about 0.1% to about 5%, of an inorganic and/or oil-insoluble sunscreen. Non-limiting examples of suitable insoluble sunscreens include methylene bis-benzotriazolyl tetramethylbutyl-phenol, titanium dioxides, zinc cerium oxides, zinc oxides, and derivatives and mixtures thereof.

Non-Polar Emollient

The composition of the present invention may comprise from about 2% to about 70%, and alternatively 30% to about 50% of a non-polar emollient. Non-limiting examples of suitable non-polar emollients include silicone oils, hydrocarbon oils, and mixtures thereof. Useful non-polar emollients in the present invention include natural, synthetic, saturated, unsaturated, straight chained, branched chained, linear, cyclic, aromatic, volatile, and non-volatile non-polar emollients, and mixtures thereof.

Non-limiting examples of suitable non-polar hydrocarbons oils include mineral oils and branched chain hydrocarbons (such as commercially available, for example, under the tradenames Permethyl™ (Permethyl Corporation™) and Isopar™ (Exxon™)). Non-limiting examples of suitable non-polar silicone oils include linear and cyclic dimethicones. Commercially available examples of these types of silicones include the Dow Corning 200 series, Dow Corning 344, and Dow Corning 345 (all available from Dow Corning™ Corp.); and SF1202, SF1204, and the Viscasil™ series (all available from the G.E. Silicones™). Additional non-polar silicone oils include alkyl (for example, 2 carbons to 30 carbons) and aryl (for example, phenyl or styrenyl) substituted silicones, including by not limited to phenyl methicone, phenyl dimethicone, phenyl trimethicone, diphenyl dimethicone, phenylethyl dimethicone, hexyl dimethicone, lauryl dimethicone, cetyl dimethicone, stearyl dimethicone, bis-stearyl dimethicone, and mixtures thereof.

Hydrophobic Components

The composition of the present invention may comprise from about 2% to about 75%, or from about 5% to about 35%, or from about 10% to about 30%, by weight of the composition, of a hydrophobic component. The hydrophobic component may be derived from animals, plants, or petroleum and may be natural or synthetic (i.e., man-made). Preferred hydrophobic components are substantially water-insoluble, more preferably essentially water-insoluble. Preferred hydrophobic components are those having a melting point of about 25° C. or less under about one atmosphere of pressure. Non-limiting examples of suitable hydrophobic components include those selected from the group consisting of mineral oil, petrolatum, esters, hydrocarbons, straight and branched chain hydrocarbons having from about 7 to about 40 carbon atoms, C1-C30 alcohol esters of C1-C30 carboxylic acids and of C2-C30 dicarboxylic acids, mono-, di- and tri-glycerides of C1-C30 carboxylic acids, alkylene glycol esters of C1-C30 carboxylic acids, propoxylated and ethoxylated derivatives, C1-C30 mono- and poly-esters of sugars and related materials, organopolysiloxane oils (polyalkylsiloxanes, cyclic polyalkylsiloxanes, trimethylsiloxysilicate, dimethiconols, polyalkylaryl siloxanes), vegetable oils and hydrogenated vegetable oils, animal fats and oils, silicone elastomers, and combinations thereof. These components are provided in further detail in U.S. Pat. No. 5,997,887 and U.S. Application Publication No. US 2005/0019356 A1.

### Soaping Index

The emulsifier system herein surprisingly avoids soaping exhibited by many personal care compositions. As discussed hereinabove, formulators have traditionally mitigated the soaping effect by using silicone, and specifically dimethicone, to minimize the appearance of a soapy film after application of the cosmetic composition. The soaping effect is measured herein via a “Soaping Index”.

The Soaping Index is based a 5-point scale according to the comparative examples discussed hereinafter. About 0.5 g of each sample formulation is applied to a user's forearm according to typical application by a consumer. The formulation which formed the greatest amount of soapy residue by visual inspection is assigned a “5”. The remaining formulations are compared visually, side-by-side, and a value is assigned on a relative basis, with “5” being about identical to the highest soapy-residue forming formulation, and a “0” being substantially free of any soapy residue.

### Method of Use

The present invention relates to a method for improving or regulating keratinous tissue condition. In one aspect, this may be accomplished by providing the personal care composition described above and applying the composition to keratinous tissue in need of treatment. Conditions to be improved or regulated include increasing the luminosity or “glow” of the skin, reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by telangiectasia or spider vessels, dryness, brittleness, and graying hair.

### Method of Making

Personal care compositions herein may be formulated according to the following steps. In a suitable vessel, the oil phase (including oil-soluble ingredients) components are combined and mixed with a suitable mixer (e.g., Tekmar RW20DZM) and heated with stirring to a temperature of about 70-80° C. and this temperature is maintained. In a separate suitable vessel, the water phase (including water-soluble ingredients) components are combined and mixed with a suitable mixer and are heated with stirring to about 70-80° C. and this temperature is maintained. The oil phase mixture is then added to the water phase mixture and mixed well so as to emulsify the combination. The emulsifier system of the oil phase and water phase components is allowed to cool to about 60° C. and then the optional ingredients are added to the emulsion with continuous mixing. The emulsion is allowed to further cool to about 40° C. The resulting emulsion is then milled using a suitable mill (e.g., with a Tekmar T-25) for about 5 minutes or until the product is uniform.

### EXAMPLES

The following examples are presented to further illustrate the compositions and methods of this invention, but are not to be construed as limiting the invention, which is delineated in the appended claims. All parts and percentages in the examples are on a weight basis and all measurements are obtained at about 23° C., unless indicated to the contrary.

Examples I-IX are formed according to the following steps. The xanthan gum and glycerin are pre-mixed in a small vessel. In a separate, larger vessel, the aloe vera gel 1X organic and water are combined, with mixing. The xanthan gum/glycerin pre-mix is added to the aloe vera gel 1X organic and water mixture in the larger vessel and mixed until uniform. The betaine and sucrose stearate are then added to the large vessel with mixing, and the vessel is heated to 80 degrees Celsius. Mixing continues in this vessel (water phase) until the contents are uniform. The methyl glucose sequisterate, helianthus annuus seed oil and butyrospermum parkii are added to a separate vessel with mixing and heated until 80 degrees (oil phase). When both the water and oil phases are uniform and the temperature is at 80 degrees Celsius, the oil phase is slowly added to the water phase with mixing. This combined mixture is then homogenized for the appropriately scaled time. The batch is then set to cool. When the temperature of the batch reaches 30-35 degrees Celsius, Geogard 221, Pheohydrane G and Tocobiol are added. The batch is then mixed until uniform and cools further to 30 degrees Celsius.

| | Example | | | | | | | | |
|---|---|---|---|---|---|---|---|---|---|
| | I. | II. | III. | IV. | V. | VI. | VII. | VIII. | IX. |
| | wt % | wt % | wt % | wt % | wt % | wt % | wt % | wt % | wt % |
| Oil phase | | | | | | | | | |
| Methyl Glucose Sequisterate¹ | 2.00 | 2.00 | 2.00 | 1.00 | 1.00 | 1.00 | 3.00 | 3.00 | 3.00 |
| helianthus annuus seed oil | 15.75 | 15.75 | 15.75 | 15.75 | 15.75 | 15.75 | 15.75 | 15.75 | 15.75 |
| butyrospermum parkii | 3.00 | 3.00 | 3.00 | 3.00 | 3.00 | 3.00 | 3.00 | 3.00 | 3.00 |
| dehydroacetic acid/benzyl | 1.00 | 1.00 | 1.00 | 1.00 | 1.00 | 1.00 | 1.00 | 1.00 | 1.00 |
| alcohol/water² | | | | | | | | | |
| Glycerin/water/hydrolyzed | 1.00 | 1.00 | 1.00 | 1.00 | 1.00 | 1.00 | 1.00 | 1.00 | 1.00 |
| algin/Chlorella vulgaris | | | | | | | | | |
| extract/sea salt³ | | | | | | | | | |
| Tocopherol⁴ | 0.10 | 0.10 | 0.10 | 0.10 | 0.10 | 0.10 | 0.10 | 0.10 | 0.10 |
| Water phase | | | | | | | | | |
| Sucrose Stearate⁵ | 2.00 | 1.50 | 5.00 | 2.00 | 1.50 | 5.00 | 2.00 | 5.00 | 0.50 |
| Aloe Vera Gel 1X Organic | 50.00 | 50.00 | 50.00 | 50.00 | 50.00 | 50.00 | 50.00 | 50.00 | 50.00 |
| xanthan gum⁶ | 0.50 | 0.50 | 0.50 | 0.50 | 0.50 | 0.50 | 0.50 | 0.50 | 0.50 |
| Glyercin | 1.60 | 1.60 | 1.60 | 1.60 | 1.60 | 1.60 | 1.60 | 1.60 | 1.60 |
| Betaine⁷ | 1.00 | 1.00 | 1.00 | 1.00 | 1.00 | 1.00 | 1.00 | 1.00 | 1.00 |

To 100% with water and/or optional ingredients
¹TEGO ® CARE PS available from Evonik Goldschmidt GmbH
²GEOGARD ™ 221 available from Lonza
³Pheohydrane G available from Codif
⁴Tocobiol ® available from BTSA
⁵Sisterna SP70-C available from Sisterna
⁶RHODICARE ® T available from Rhodia Novecare
⁷Betafin BP-20

### Comparative Examples

The formulation of Example I above is compared to a Comparative Samples A, B, and C, respectively. The Comparative Sample C, which contains no dimethicone, has a high soap index when applied to skin. As demonstrated below, increasing levels of dimethicone reduce the incidence of high soaping. The Comparative Sample A achieves parity to Example I, with respect to its Soapiness Index score, when it includes 3% of dimethicone. In contrast, Example I is free of silicone emulsifiers.

| | Comparative Sample | | |
|---|---|---|---|
| | A | B | C |
| Oil phase | | | |
| Cetearyl olivate/sorbitan olivate⁸ | 4.00 | 4.00 | 4.00 |
| Helianthus annuus seed oil | 15.75 | 15.75 | 15.75 |
| Butyrospermum parkii | 3.00 | 3.00 | 3.00 |
| Dehydroacetic acid/benzyl alcohol/ | 1.00 | 1.00 | 1.00 |
| water⁹ | | | |
| Glycerin/water/hydrolyzed algin/ | 1.00 | 1.00 | 1.00 |
| Chlorella vulgaris extract/sea salt¹⁰ | | | |
| Tocopherol¹¹ | 0.10 | 0.10 | 0.10 |
| Dimethicone¹² | 3.00 | 0.50 | 0.00 |
| Water phase | | | |
| Aloe Vera Gel 1X Organic | 50.00 | 50.00 | 50.00 |
| Xanthan gum¹³ | 0.50 | 0.50 | 0.50 |
| Glyercin | 1.60 | 1.60 | 1.60 |
| Betaine¹⁴ | 1.00 | 1.00 | 1.00 |
| Water | 19.05 | 19.05 | 19.05 |

⁸Olivem ® 1000
⁹GEOGARD ™ 221 available from Lonza
¹⁰Pheohydrane G available from Codif
¹¹Tocobiol ® available from BTSA
¹²Xiameter PMX-200 Silicone FL. 10CS available from Dow Corning
¹³RHODICARE ® T available from Rhodia Novecare
¹⁴Betafin BP-20

| Formulation | Soapiness Index |
|---|---|
| Example I | 1 |
| Comparative Sample A | 1 |
| Comparative Sample B | 3 |
| Comparative Sample C | 5 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention, as are ranges based thereon. Where a combination is disclosed, each subcombination of the elements of that combination is also specifically disclosed and is within the scope of the invention. Conversely, where different elements or groups of elements are disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic composition comprising at least one sucrose fatty acid ester and at least one nonionic surfactant, said composition being substantially free of silicone emulsifiers.

2. A personal care composition according to claim 1, wherein said sucrose fatty acid ester is selected from the group consisting of sucrose dilaurate, sucrose distearate, sucrose hexaerucate; sucrose hexaoleate/hexapalmitate/hexastearate, sucrose hexapalmitate, sucrose laurate; sucrose myristate, sucrose oleate, sucrose palmitate, sucrose pentaerucate, sucrose polybehenate, sucrose polylaurate, sucrose polylinoleate, sucrose polyoleate, sucrose polystearate, sucrose ricinoleate, sucrose stearate, sucrose tetraisostearate, sucrose tribehenate, sucrose trilaurate, sucrose tristearate, sucrose cocoate, sucrose polycottonseedate, sucrose polypalmate, sucrose polysoyate, and mixtures thereof.

3. A personal care composition according to claim 1, wherein said nonionic surfactant is selected from the group consisting of methyl glucose sesquisterate, methyl glucose ether, methyl glucose ether distearate, PEG-120 methyl glucose dioleate and ethoxylated methyl glucose having from about 10 to about 20 repeating ethoxy units.

4. A personal care composition according to claim 1, wherein said sucrose fatty acid ester is sucrose stearate and said nonionic surfactant is methyl glucose sesquisterate.

5. A personal care composition emulsion according to claim 1, wherein said sucrose stearate and said methyl glucose sesquisterate are comprised at a ratio ranging from about 1:3 to about 3:1.

6. A personal care composition according to claim 1, further comprising an active selected from the group consisting of actives include sugar amines, vitamin B3 compounds, vitamins, peptides, sunscreens, retinoids, and mixtures thereof.

7. A personal care composition according to claim 1, further comprising at least one active selected from the group consisting of oil control agents, N-acyl amino acid compounds, tanning actives, anti-acne actives, desquamation actives, anti-cellulite actives, chelating agents, skin lightening agents, flavonoids, protease inhibitors, tyrosinase inhibitors, non-vitamin antioxidants and radical scavengers, preservatives, hair growth regulators, anti-wrinkle actives, anti-atrophy actives, minerals, phytosterols and/or plant hormones, anti-inflammatory agents, antimicrobials, antifungals, and mixtures thereof.

8. A personal care composition according to claim 1, further comprising one or more actives selected from the group consisting of caffeine, tea extract, ginseng, cucumber extract, rosehip oil, date palm kernel extract, witch hazel extract, dill extract, tetrahydrocurcmin; turmerone, and mixtures thereof.

9. A personal care composition according to claim 1, further comprising one or more particulate materials.

10. A personal care composition according to claim 1, further comprising at least one emollient.

11. A cosmetic composition comprising an emulsifier system premix, said premix consisting of a sucrose fatty acid ester and a nonionic surfactant; wherein said composition is substantially free of silicone emulsifiers.
